(19) United States
(12) Patent Application Publication
(10) Pub. No.: US 2007/0116741 A1
(43) Pub. Date: May 24, 2007

(54) Contact lenses with mucin affinity

(75) Inventors: Paul L. Valint JR., Pittsford, NY (US); Joseph A. McGee, Dewitt, NY (US); David P. Vanderbilt, Webster, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/284,025

(22) Filed: Nov. 21, 2005

(51) Int. Cl. A61F 2/02

(52) U.S. Cl. 424/428

(57) ABSTRACT

A biomedical device surface, such as a contact lens surface, is covalently linked to a cationic copolymer. The copolymer may include monomeric units derived from an ethylenically unsaturated monomer containing a quaternary ammonium moiety and monomeric units derived from an ethylenically unsaturated monomer containing a moiety reactive with complementary reactive functionalities at the lens surface. The copolymer may be covalently linked directly to the lens surface, or covalently linked with an intermediate polymer reactive with both the lens surface and the copolymer.

### BACKGROUND OF THE INVENTION

Mucins are glycoconjugated proteins which are secreted by vesicles and discharged on the surface of the conjunctival epithelium of the eye. Mucins are found on moist, mucosal epithelia, and are thought to combine mechanical protection of eye tissue as well as chemical and immune protection of mucosal tissue. The surface of the eye is kept moist and lubricated by tear film. Mucins anchor this tear film to the epithelium and protect the eye surface from bacterial, chemical and physical invasion of foreign bodies.

U.S. Pat. Nos. 6,348,508 (Denick, Jr. et al.), 2004/0063620 (Xia et al.), and 2004/0063591 (Borazjani et al.) disclose compositions for treating dry eye or for treating contact lenses that comprise a cationic polysaccharide. In the case of eye drop solutions, the cationic polysaccharides, after binding to the mucosal eye tissue, may in turn promote the mucins in the eye, either by supplementing the mucin and/or by helping to bind and maintain mucin on the eye surface.

In the case of contact lenses, mucins are often viewed as a debris that, like other proteins, should not accumulate on the contact lens surface. For example, U.S. Pat. No. 5,985,629 (Aaslyng et al.) discloses contact lens cleaning and disinfecting compositions comprising an enzyme and an enzyme inhibitor. As another example, U.S. Pat. No. 6,649,722 (Rosenzweig et al.) discloses contact lens compositions. At column 28, it is reported that binding of mucin to the lens was at a desirably low enough level that the mucin would not lead to corneal adhesion of the lens.

### SUMMARY OF THE INVENTION

According to various embodiments, this invention provides a biomedical device, such as a contact lens, comprising a surface covalently linked to a cationic copolymer. Preferably, the copolymer comprises monomeric units derived from an ethylenically unsaturated monomer containing a quaternary ammonium moiety. The copolymer may comprise hydrophilic, cationic monomeric units and/or hydrophobic, cationic monomeric units. The copolymer may further comprise monomeric units derived from a non-cationic, ethylenically unsaturated hydrophilic monomer, and/or monomeric units derived from an ethylenically unsaturated monomer containing a moiety reactive with complementary reactive functionalities at the lens surface.

The copolymer may be covalently linked directly to the device surface with the moiety reactive with the complementary functionality at the device surface, or covalently linked with an intermediate polymer reactive with both the device surface and the copolymer.

According to other embodiments, the cationic copolymer on the device surface complexes with mucin. In the case of ophthalmic devices, the cationic copolymer complexes with epithelial mucin when placed on the eye.

The invention also provides a method comprising covalently linking to a biomedical device surface a cationic copolymer, for example, the copolymer comprises reactive moieties that are covalently linked to complementary reactive functionalities on the device surface. The device surface may be subject to a plasma treatment that provides the complementary reactive functionalities on the device surface. An intermediate polymer may be linked to the device surface, and the cationic copolymer is linked to this intermediate polymer.

This invention includes a method comprising placing a contact lens with the cationic polymer on its surface in the eye, whereby mucin complexes with the cationic copolymer. Various copolymers are provided by this invention.

### DETAILED DESCRIPTION

This invention is useful for biomedical devices. The term “biomedical device” denotes a device that is placed into contact with tissue. This invention is especially useful for biomedical devices placed into contact with mucosal tissue. According to various preferred embodiments, the biomedical device is an ophthalmic device, intended for placement in contact with epithelial tissue, especially corneal onlays and contact lenses. The following disclosure references contact lenses, but is applicable to various other biomedical devices.

This invention is useful for all known types of contact lenses, including both soft and rigid lens materials. Hydrogels represent one class of materials used for contact lens applications. Hydrogels comprise a hydrated, cross-linked polymeric system containing water in an equilibrium state. Accordingly, hydrogels are copolymers prepared from hydrophilic monomers. In the case of silicone hydrogels, the hydrogel copolymers are generally prepared by polymerizing a mixture containing at least one device-forming silicone-containing monomer and at least one device-forming hydrophilic monomer. Either the silicone-containing monomer or the hydrophilic monomer may function as a crosslinking agent (a crosslinking agent being defined as a monomer having multiple polymerizable functionalities), or alternately, a separate crosslinking agent may be employed in the initial monomer mixture from which the hydrogel copolymer is formed. (As used herein, the term “monomer” or “monomeric” and like terms denote relatively low molecular weight compounds that are polymerizable by free radical polymerization, as well as higher molecular weight compounds also referred to as “prepolymers”, “macromonomers”, and related terms.) Silicone hydrogels typically have a water content between about 10 to about 80 weight percent.

Examples of useful lens-forming hydrophilic monomers include: amides such as N,N-dimethylacrylamide and N,N-dimethylmethacrylamide; cyclic lactams such as N-vinyl-2-pyrrolidone; (meth)acrylated alcohols, such as 2-hydroxyethyl methacrylate and 2-hydroxyethylacrylate; and (meth)acrylated poly(ethyleneglycol)s; and azlactone-containing monomers, such as 2-isopropenyl-4,4-dimethyl-2-oxazolin-5-one and 2-vinyl-4,4-dimethyl-2-oxazolin-5-one. (As used herein, the term “(meth)” denotes an optional methyl substituent. Thus, terms such as “(meth)acrylate” denotes either methacrylate or acrylate, and “(meth)acrylic acid” denotes either methacrylic acid or acrylic acid.) Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277, the disclosures of which are incorporated herein by reference. Other suitable hydrophilic monomers will be apparent to one skilled in the art.

As mentioned, one preferred class hydrogel contact lens materials is silicone hydrogels. In this case, the initial lens-forming monomer mixture further comprises a silicone-containing monomer.

Applicable silicone-containing monomeric materials for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

Examples of applicable silicon-containing monomers include bulky polysiloxanylalkyl (meth)acrylic monomers. An example of bulky polysiloxanylalkyl (meth)acrylic monomers are represented by the following Formula I:

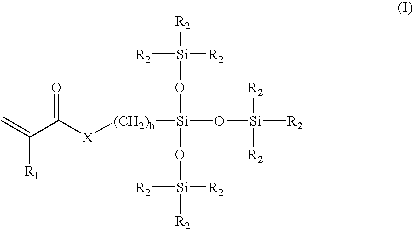

wherein:

X denotes —O— or —NR—;

each R₁ independently denotes hydrogen or methyl;

each R₂ independently denotes a lower alkyl radical, phenyl radical or a group represented by

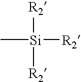

wherein each R₂′ independently denotes a lower alkyl or phenyl radical; and h is 1 to 10. One preferred bulky monomer is methacryloxypropyl tris(trimethylsiloxy)silane or tris(trimethylsiloxy)silylpropyl methacrylate, sometimes referred to as TRIS.

Another class of representative silicon-containing monomers includes silicone-containing vinyl carbonate or vinyl carbamate monomers such as: 1,3-bis[4-vinyloxycarbonyloxy)but-1-yl]tetramethyl-disiloxane; 1,3-bis[4-vinyloxycarbonyloxy)but-1-yl]polydimethylsiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]-propyl vinyl carbamate; 3-[tris(trimethylsiloxy)silyl]-propyl allyl carbamate; 3-[tris(trimethylsiloxy)silyl]propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula II: 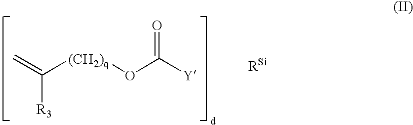
wherein:

Y′ denotes —O—, —S— or —NH—;

R^{Si} denotes a silicone-containing organic radical;

R₃ denotes hydrogen or methyl;

d is 1,2, 3 or 4; and q is 0 or 1.

Suitable silicone-containing organic radicals R^{Si} include the following:
—(CH₂)_{n′}Si[(CH₂)_{m′}CH₃]₃;
—(CH₂)_{n′}Si[OSi(CH₂)_{m′}CH₃]₃; 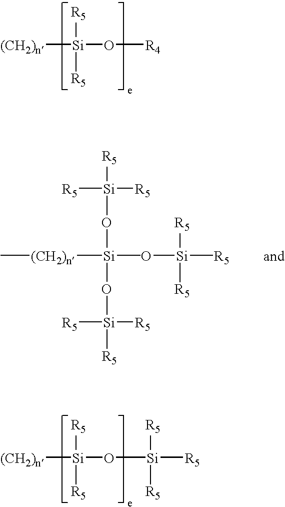
wherein:

R₄ denotes 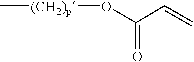
wherein p′ is 1 to 6;

R₅ denotes an alkyl radical or a fluoroalkyl radical having 1 to 6 carbon atoms;

e is 1 to 200; n′ is 1, 2, 3 or 4; and m′ is 0, 1, 2, 3, 4 or 5.

An example of a particular species within Formula II is represented by Formula III:

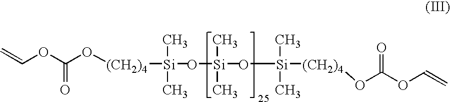

Another class of silicon-containing monomers includes polyurethane-polysiloxane macromonomers (also sometimes referred to as prepolymers), which may have hard-soft-hard blocks like traditional urethane elastomers. Examples of silicone urethane monomers are represented by Formulae IV and V:
E(*D*A*D*G)ₐ*D*A*D*E′; or (IV)
E(*D*G*D*A)ₐ*D*G*D*E′ (V);
wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureido linkage;

a is at least 1;

A denotes a divalent polymeric radical of Formula VI: 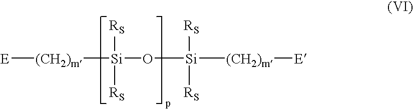
wherein:
each Rₛ independently denotes an alkyl or fluoro-substituted alkyl group having 1 to 10 carbon atoms which may contain ether linkages between carbon atoms;
m′ is at least 1; and
p is a number which provides a moiety weight of 400 to 10,000;
each of E and E′ independently denotes a polymerizable unsaturated organic radical represented by Formula VII: 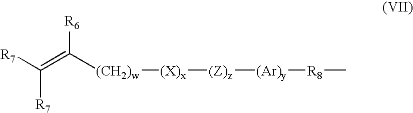
   wherein:

R₆ is hydrogen or methyl;

R₇ is hydrogen, an alkyl radical having 1 to 6 carbon atoms, or a —CO—Y—R₉ radical wherein Y is —O—, —S— or —NH—;

R₈ is a divalent alkylene radical having 1 to 10 carbon atoms;

R₉ is a alkyl radical having 1 to 12 carbon atoms;

X denotes —CO— or —OCO—;

Z denotes —O— or —NH—;

Ar denotes an aromatic radical having 6 to 30 carbon atoms;

w is 0 to 6; x is 0 or 1; y is 0 or 1; and z is 0 or 1.

A more specific example of a silicone-containing urethane monomer is represented by Formula (VIII):

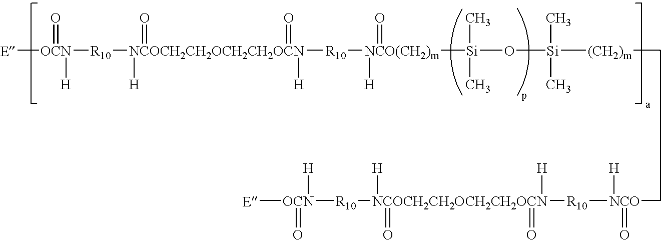

wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

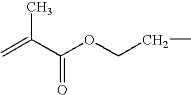

A preferred silicone hydrogel material comprises (based on the initial monomer mixture that is copolymerized to form the hydrogel copolymeric material) 5 to 50 percent, preferably 10 to 25, by weight of one or more silicone macromonomers, 5 to 75 percent, preferably 30 to 60 percent, by weight of one or more polysiloxanylalkyl (meth)acrylic monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

An additional class of contact lens materials are rigid copolymers, especially rigid, gas-permeable (RGP) copolymers. RGP copolymers generally include: a silicone-containing monomer, including any of the aforementioned silicone-containing monomers mentioned above; a hydrophilic monomer as a wetting agent; a hardness modifying monomer; and a crosslinking agent; a polymerization initiator; an ultraviolet blocking agent; or a colorant.

Specific examples of contact lens materials useful in the present invention are taught in U.S. Pat. Nos. 6,891,010 (Kunzler et al.); 5,908,906 (Kunzler et al.); 5,714,557 (Kunzler et al.); 5,710,302 (Künzler et al.); 5,708,094 (Lai et al.); 5,616,757 (Bambury et al.); 5,610,252 (Bambury et al.); 5,512,205 (Lai); 5,449,729 (Lai); 5,387,662 (Kunzler et al.); 5,310,779 (Lai); 5,260,000 (Nandu et al.); and 5,346,976 (Ellis et al.); the disclosures of which are incorporated herein by reference.

The copolymers linked to the contact lens surface contain cationic moieties that complex, or form a complex, with mucin. More specifically, the cationic moieties are able to complex with the polysaccharide side chains found in mucin, and thereby possess an affinity for the mucins in tear fluid while the contact lens is worn.

Preferred copolymers contain cationic, quaternary nitrogen moieties. Especially, the copolymers may comprise monomeric units derived from an ethylenically unsaturated monomer containing the cationic, quaternary nitrogen moieties.

The monomeric units may be derived from hydrophilic, ethylenically unsaturated monomers containing a quaternary nitrogen atom. Preferred are (meth)acrylate or (meth)acrylamide monomers containing a radical of formula (X):
—(R¹¹)—N⁺(R¹²)(R¹³)(R¹⁴) (X)
wherein:

R¹¹ is C(1-3) alkylene; and

each of R¹², R¹³ and R¹⁴ is independently methyl or ethyl. Examples include: methyl and ethyl halide salts of 2-(N,N-dimethyl)-ethylamino(meth)acrylates, 2-(N,N-dimethyl)-ethylamino(meth)acrylamides, 3-(N,N-dimethyl)-propylamino(meth)acrylates, and 3-(N,N-dimethyl)-propylamino(meth)acrylamides.

The monomeric units may be derived from hydrophobic, ethylenically unsaturated monomers containing a quaternary nitrogen atom. Preferred are (meth)acrylate or (meth)acrylamide monomers containing a radical of formula (XI):
—(R²¹)—N⁺(R²²)(R²³)(R²⁴) (XI)
wherein:

R²¹ is C(1-10) alkylene or fluoroalkylene;

each of R²² and R²³ is independently methyl or ethyl; and

R²⁴ is C(4-22) alkyl or fluoroalkyl. Specific examples include: C(8-18) alkyl salts of N,N-dimethyl-ethylamino(meth)acrylates, N,N-dimethyl-ethylamino(meth)acrylamides, N,N-dimethyl-propylamino(meth)acrylates, and N,N-dimethyl-propylamino(meth)acrylamides.

According to preferred embodiments, the cationic copolymer includes monomeric units derived from both formulae (X) and (XI). The hydrophilic, cationic monomeric units have affinity to mucin, and since they are hydrophilic, they are readily soluble in aqueous media. The hydrophobic, cationic monomeric units have affinity to mucin but are less soluble in aqueous media; in some cases, it is possible these copolymers comprising the hydrophobic, cationic monomeric units of formula (XI) provide copolymers with better affinity to epithelial mucin than copolymers containing only hydrophilic, cationic monomeric units.

The preferred copolymers include, in addition to the monomeric units derived from a cationic, ethylenically unsaturated monomer, a monomeric unit derived from an ethylenically unsaturated monomer containing a reactive moiety. Specifically, the ethylenic unsaturation of this monomer renders the monomer copolymerizable with the cationic monomer. In addition, this monomer contains the reactive moiety that is reactive with complementary reactive functionalities at the lens surface, and/or complementary reactive functionalities of an intermediate polymer, discussed in more detail below.

Examples of reactive monomers include: ethylenically unsaturated carboxylic acids, such as (meth)acrylic acid; ethylenically unsaturated primary amines, such as ethylamino(meth)acrylate, ethylamino(meth)acrylamide, propylamino(meth)acrylate, and propylamino(meth)acrylamide; alcohol-containing (meth)acrylates and (meth)acrylamides, such as 2-hydroxyethyl methacrylate; ethylenically unsaturated epoxy-containing monomers, such as glycidyl methacrylate or glycidyl vinyl carbonate; and azlactone-containing monomers, such as 2-isopropenyl-4,4-dimethyl-2-oxazolin-5-one and 2-vinyl-4,4-dimethyl-2-oxazolin-5-one, where the azlactone group hydrolyzes in aqueous media to convert the oxazolinone moiety to a reactive carboxylic acid moiety.

The copolymers may further include a non-cationic hydrophilic monomeric unit. Examples include ethylenically unsaturated monomers that are copolymerizable with the cationic, ethylenically unsaturated monomer. Specific examples include: N,N-dimethylacrylamide and N,N-dimethylmethacrylamide; cyclic lactams such as N-vinyl-2-pyrrolidone; (meth)acrylated alcohols, such as 2-hydroxyethyl; methacrylate and 2-hydroxyethylacrylate; and (meth)acrylated poly(ethyleneglycol)s. The main purpose of the hydrophilic monomeric unit in the polymer, when used, is to ensure the polymer is readily water-soluble, thus avoiding the need to dissolve the polymer in organic solvent when applying the polymer to the lens surface.

The copolymers may further include a non-cationic hydrophobic monomeric unit. Examples include: ethylenically unsaturated monomers comprising (C8-C20) alkyl, including (meth)acrylate alkyl and (meth)acrylamide alkyl; ethylenically unsaturated monomers comprising (C1-C20) fluoroalkyl, including (meth)acrylate fluoroalkyl, such as octafluoropentylmethacrylate, and (meth)acrylamide fluoroalkyl; and ethylenically unsaturated monomers comprising cycloalkyl groups, such as cyclohexylmethacrylate.

Accordingly, preferred copolymers comprise: monomeric units derived from an ethylenically unsaturated monomer containing cationic, quaternary nitrogen moieties; and monomeric units derived from an ethylenically unsaturated monomer containing a moiety reactive with complementary reactive functionalities at the lens surface. Especially preferred are copolymers containing hydrophilic, cationic monomeric units and hydrophobic, cationic monomeric units. These copolymers may further include monomeric units derived from an ethylenically unsaturated hydrophilic monomer in an amount sufficient to render the copolymer water soluble.

Preferably, the copolymers comprise: 1 to 30 mole percent of the quaternary nitrogen-containing monomeric units, more preferably 2 to 20 mole percent; and 2 to 50 mole percent of monomeric units derived from an ethylenically unsaturated monomer containing the moiety reactive with complementary reactive functionalities at the lens surface, more preferably 5 to 40 mole percent. Preferably, these copolymers comprise 0 to 90 mole percent of the hydrophilic monomeric units, more preferably 20 to 80 mole percent. Preferably, the copolymers comprise: 1 to 30 mole percent of the hydrophilic, quaternary nitrogen-containing monomeric units, more preferably 2 to 20 mole percent; and 0 to 30 mole percent of the hydrophobic, quaternary nitrogen-containing monomeric units, more preferably 2 to 20 mole percent.

As mentioned, the copolymers include monomeric units derived from an ethylenically unsaturated monomer containing a reactive moiety, and this reactive moiety links the polymer to the lens surface. One manner of linking the cationic copolymer to the lens surface involves forming the lens from a monomer mixture including a monomer that includes reactive functionalities that are complementary with the reactive moiety of the cationic copolymer.

As a first example, the contact lens may be formed of the polymerization product of a monomer mixture comprising an epoxy-containing monomer, such as glycidyl methacrylate or glycidyl vinyl carbonate. Sufficient epoxy groups will migrate to the lens surface, and these epoxy groups covalently react with functionalities of the cationic copolymer, especially carboxylic acid, amino and alcohol reactive moieties.

As a second example, the contact lens may be formed of the polymerization product of a monomer mixture comprising a carboxylic acid-containing monomer, such as (meth)acrylic acid or vinyl carbonic acid. Sufficient carboxylic groups will be present at the lens surface to covalently react with functionalities of the cationic copolymer, especially amino and alcohol reactive moieties.

As a third example, the contact may be formed of the polymerization product of a monomer mixture comprising an azlactone-containing monomer, such as 2-isopropenyl-4,4-dimethyl-2-oxazolin-5-one and 2-vinyl-4,4-dimethyl-2-oxazolin-5-one. Azlactone groups at the lens surface will hydrolyze in aqueous media to convert the oxazolinone group to a carboxylic acid, for reaction with the cationic co polymer reactive moieties.

As another example, the contact lens may be formed of the polymerization product of a monomer mixture comprising a (meth)acrylate or (meth)acrylamide alcohol, such as 2-hydroxyethyl methacrylate. The alcohol groups are available to react with cationic polymer reactive moieties.

Other lens-forming monomers containing complementary reactive groups are known in the art, including those disclosed in U.S. Pat. No. 6,440,571 (Valint, Jr. et al.), the entire disclosure of which is incorporated herein by reference.

Another manner of linking the cationic copolymer to the lens surface involves treating the lens surface to provide reactive functionalities that are complementary with the reactive moiety of the cationic copolymer. As an example, the lens surface may be subjected to plasma treatment in an oxygen-containing atmosphere to form alcohol functionalities on the lens surface, or in a nitrogen-containing atmosphere to form amine functionalities on the lens surface. In the case that the contact lens contains fluorine at its surface, the lens surface may be initially plasma treated in a hydrogen atmosphere to reduce fluorine content at the lens surface. Such methods are known in the art, including U.S. Pat. Nos. 6,550,915 and 6,794,456 (Grobe III), the entire disclosures of which are incorporated herein by reference.

The alcohol or amino functionality generated at the lens surface by the plasma treatment may then react with reactive moieties of the cationic copolymer, especially carboxylic acid moieties, amino and alcohol reactive moieties.

A variation of plasma treatment involves initially subjecting the lens surface to a plasma oxidation, followed by plasma polymerization in an atmosphere containing a hydrocarbon (such as a diolefin, for example, 1,3-butadiene) to form a carbon layer on the lens surface. Then, this carbon layer is plasma treated in an oxygen or nitrogen atmosphere to generate hydroxyl or amine radicals. The reactive moiety of the cationic copolymer can then be covalently attached to the hydroxyl or amine radicals of the carbon layer. This method is disclosed in U.S. Pat. No. 6,213,604 (Valint, Jr. et al.), the entire disclosure of which is incorporated herein by reference.

As used herein, the term “plasma treatment” is inclusive of wet or dry corona discharge treatments.

Another manner of linking the cationic copolymer to the lens surface involves employing an intermediate polymer. More specifically, the intermediate polymer is linked to both the cationic copolymer and the lens surface. Thus, this intermediate polymer has functionality reactive with the lens surface, as well as functionality reactive with the reactive moieties of the cationic copolymer.

This intermediate polymer may be covalently linked to the lens surface by the various methods, discussed supra in relation to direct linking of the cationic copolymer. For example, the contact lens may be formed of a monomer mixture including a monomer that includes reactive functionalities that are complementary with the reactive functionalities of the intermediate polymer. Alternately, the contact lens surface may be treated, for example, plasma treated, to provide reactive sites for the intermediate polymer

The intermediate polymer may be covalently linked to the cationic copolymer by providing both polymers with complementary reactive groups, including those mentioned supra. Additional examples are found in U.S. Pat. No. 6,440,571 (Valint, Jr. et al.).

As an example, the lens may be coated with a mixture of an intermediate copolymer of dimethylacrylamide and glycidyl methacrylate, and a cationic copolymer. The epoxy functionality of the intermediate copolymer will covalently link to hydroxyl, primary amine or carboxylic acid moieties at the lens surface, and will covalently link to hydroxyl, primary amine or carboxylic acid moieties of the cationic copolymer. Numerous other examples of intermediate polymers are evident.

Accordingly, various methods generally known in the art are available for linking the cationic copolymer to the contact lens surface. Other methods will be evident to ones skilled in the art.

The following examples illustrate various preferred embodiments of this invention.

### EXAMPLE 1

### Synthesis of Methyl Iodide Salt of DMAPMA

To a 1-L 3-neck round-bottom flask equipped with a mechanical stirrer, addition funnel and drying tube was added 62.66 g iodomethane (0.44 mol) and 650-mL of diethyl ether. The addition funnel was charged with a solution of 68.10 g 3-(N,N-dimethylamino)propyl methacrylamide (DMAPMA) in 250 mL diethyl ether. The contents of the addition funnel were added dropwise to the stirred contents over 1 to 2 hours. The resultant slurry was stirred overnight and the precipitate was collected by vacuum filtration. The white solid was dried in vacuo at 40° C. giving 113.6 g of product (91% yield).

### EXAMPLE 2

### Synthesis of Dodecyl Iodide Salt of DMAPMA

To a 1-L 3-neck round-bottom flask equipped with a magnetic stir bar, addition funnel and drying tube was added 64.20 g iodododecane (0.22 mol) and 100-mL of dry tetrahydrofuran (THF). The addition funnel was charged with a solution of 34.05 g 3-(N,N-dimethylamino)propyl methacrylamide (DMAPMA) in 100-mL dry THF. The contents of the addition funnel were added dropwise to the stirred contents over 1 hour. The resultant solution was stirred for 6 days at room temperature. The flask contents were added dropwise to 2-L of stirred diethyl ether to precipitate the product. The precipitate was isolated by vacuum filtration. The white solid was dried in vacuo at 40° C. for 2 days giving 57.4 g of product (61% yield).

### EXAMPLE 3

### Synthesis of Cationic Polymer

To a 1-L 3-neck round bottom flask containing a magnetic stir bar, water-cooled condenser and thermocouple is added approximately 0.2 wt % AIBN initiator (based on total weight of monomers), 7.5 mol % of the methyl iodide salt of 3-(N,N-dimethylamino)propyl methacrylamide (DMAPMA-C1), 2.5 mol % of the dodecyl iodide salt of 3-(N,N-dimethylamino)propyl methacrylamide e (DMAPMA-C12), 20-mol % of glycidyl methacrylate (GMA) and 70-mol % of N,N-dimethylacrylamide (DMA). The monomers and initiator are dissolved by addition of 300-mL of methanol to the flask. The solution is sparged with argon for at least 10-min. before gradual heating to 60° C. Sparging is discontinued when the solution reaches 40 to 45° C. and the flask is subsequently maintained under argon backpressure. Heating is discontinued after 48 to 72 hours at which point the cooled solution is added drop wise to 5 L of mechanically stirred ethyl ether. The precipitate is isolated either by filtration or decanting off the ether. The solid is dried in vacuo at 40° C. for a minimum of 18 hours, and reprecipitated by dissolution in 300-mL methanol and dropwise addition into 5-L of stirred ethyl ether. The final polymer mass is determined after vacuum drying at 40° C. to a constant mass.

### EXAMPLES 4-12

### Synthesis of Cationic Polymers

The polymers in Table 1 were synthesized according to the general procedure of Example 3, by varying the molar amounts of the various monomers. In Example 12, dodecylmethacrylate (lauryl methacrylate, LMA) was included in the reaction mixture as a hydrophobic monomer.

TABLE 1

| | Example 3 | Example 4 | Example 5 | Example 6 | Example 7 |
|---|---|---|---|---|---|
| DMA (mol %) | 70 | 70 | 85 | 72.5 | 71.25 |
| GMA (mol %) | 20 | 20 | 10 | 15 | 15 |
| DMAPMA-C1 | 7.5 | 5 | 2.5 | 7.5 | 10 |
| (mol %) | | | | | |
| DMAPMA-C12 | 2.5 | 5 | 2.5 | 5 | 3.75 |
| (mol %) | | | | | |

| | Example 8 | Example 9 | Ex 10 | Ex 11 | Ex 12 |
|---|---|---|---|---|---|
| DMA (mol %) | 70 | 75 | 75 | 67.5 | 70 |
| GMA (mol %) | 15 | 15 | 15 | 12.5 | 20 |
| DMAPMA-C1 | 10 | 10 | — | 12.5 | 7.5 |
| (mol %) | | | | | |
| DMAPMA-C12 | 5 | — | 10 | 7.5 | — |
| (mol %) | | | | | |
| LMA (mol %) | — | — | — | — | 2.5 |

### EXAMPLE 13

### Coating of Contact Lenses with Cationic Polymers

Contact lenses made of balafilcon A were cast and stored in borate buffer solution (BBS). Balafilcon A is a copolymer comprised of 3-[tris(trimethylsiloxy)silyl] propyl vinyl carbamate, N-vinyl-2-pyrrolidone (NVP), 1,3-bis[4-vinyloxycarbonyloxy)but-1-yl]polydimethylsiloxane and N-vinyloxycarbonyl alanine. The lenses were not plasma treated, and these lenses are designated as “No Plasma Control” in the following tables.

Some lenses of this batch were desalinated in deionized water, dried and subjected to successive plasma regimens of ammonia, butadiene and ammonia (ABA). Some lenses retained as further controls are designated “ABA Control” in the following tables.

For coating with the subject cationic polymers, each ABA treated lens was placed in a glass scintillation vial containing 3-mL of a 2% (w/v) solution of cationic polymer dissolved in deionized water. The vials were capped and placed in a forced-air oven heated to 90° C. for 2 hours. After cooling, the coating solution was removed by aspiration and replaced with 20-mL of deionized (DI) water with shaking. After two additional aspiration/irrigation cycles, the lenses were sealed in polypropylene contact lens blister packs in BBS. The blister packs were autoclaved at 121° C. for 30-min.

Table 2 reports various surface properties of several coated samples and controls. Polymer Examples 4, 3 and 5 were used for Coated Samples 1, 2 and 3, respectively. Atomic concentrations were determined by XPS, as described below. Contact angle was determined as described below.

TABLE 2

| | | | | | Contact |
|---|---|---|---|---|---|
| | XPS Atomic Concentrations | | | | Angle |
| | % C | % O | % N | % Si | (Water) |
| Coated | 70.2 +/− 1.7 | 18.3 +/− 1.3 | 9.2 +/− 0.6 | 2.4 +/− 0.5 | 74 +/− 6 |
| Sample 1 | | | | | |
| ABA | 66.1 +/− 2.7 | 19.2 +/− 1.3 | 8.4 +/− 0.6 | 6.4 +/− 1.2 | 94 +/− 4 |
| Control 1 | | | | | |
| Coated | 69.4 +/− 2.9 | 18.5 +/− 1.2 | 9.3 +/− 0.7 | 2.8 +/− 1.3 | 72 +/− 8 |
| Sample 2 | | | | | |
| ABA | 68.5 +/− 3.1 | 17.2 +/− 1.6 | 9.7 +/− 0.8 | 4.6 +/− 1.2 | 94 +/− 6 |
| Control 2 | | | | | |
| Coated | 67.2 +/− 3.9 | 18.6 +/− 1.7 | 11.1 +/− 0.7 | 3.2 +/− 1.5 | 79 +/− 9 |
| Sample 3 | | | | | |
| ABA | 65.0 +/− 3.9 | 19.9 +/− 1.8 | 8.7 +/− 0.9 | 6.5 +/− 1.7 | 90 +/− 8 |
| Control 3 | | | | | |
| No Plasma | 54.8 +/− 0.8 | 24.0 +/− 0.3 | 8.2 +/− 0.6 | 13.1 +/− 1.1 | 115 +/− 1 |
| Control | | | | | |

X-ray Photoelectron Spectroscopy (XPS) Analysis

XPS data was collected using a Physical Electronics Quantera SXM Scanning ESCA Microprobe. This instrument utilizes a monochromatic A1 anode operated at 18 kV and 100 Watts in the high power mode and 15 kV and 0.25 Watts/micron in low power mode. All high power acquisitions are rastered over a 1400 micron x 100 micron analysis area. Dual beam neutralization (ions and electrons) is used. The base pressure of the instrument was 5×10⁻¹⁰ torr and during operation the pressure was less than or equal to 1×10⁻⁷ torr. This instrument made use of a hemispherical analyzer operated in FAT mode. A gauze lens was coupled to a hemispherical analyzer in order to increase signal throughput. Assuming the inelastic mean free path for a carbon 1s photoelectron is 35 Å, the practical measure for sampling depth for this instrument at a sampling angle of 45 is approximately 75 Å. The governing equation for sampling depth in XPS is:
θλsin3=d
where d is the sampling depth, λ is the photoelectron inelastic mean free path and θ is the angle formed between the sample surface and the axis of the analyzer. Each specimen was analyzed utilizing a low-resolution survey spectra (0-1100eV) to identify the elements present on the sample surface. Quantification of elemental compositions was completed by integration of the photoelectron peak areas. Analyzer transmission, photoelectron cross-sections and source angle correction were taken into consideration in order to give accurate atomic concentration values.
Contact Angle Analysis

The instrument used for measurement was a Video Contact Angle System (VCA) 2500XE, (AST Products, Inc., Billerica, Mass., USA). This instrument utilizes a low-power microscope that produces a sharply defined image of the water drop, which is captured immediately on the computer screen. HPLC water is drawn into the VCA system microsyringe, and a 0.6 μl drop is dispensed from the syringe onto the sample. The contact angle is calculated by placing five markers along the circumference of the drop. The software of the system calculates a curve representing the circumference of the drop and the contact angle is recorded.

### EXAMPLE 14

### Mucin Affinity

Mucin affinity was evaluated using an enzyme linked lectin assay. This assay utilizes biotinylated jacalin as a probe for detection of mucin on the contact lens surface. The strong biotin-streptavidin interaction provides the base for further signal amplification using a streptavidin-peroxidase conjugate.

Coated Samples 1, 2 and 3 from Example 13 were evaluated, as well as two controls, PV Control and No Plasma Control from Example 13.

To test the mucin affinity of the contact lens material, purified Bovine Submaxillary Gland Mucin (BSM) was used. The mucin solution was prepared at 0.5 mg/ml using a 20 mM PBS buffer (PBS20; pH 7.4; Na/K=33). The contact lenses were stored at room temperature prior to analysis. First, the lenses were washed with PBS20 and transferred with a tweezer to a vial containing the mucin solution. Incubation with the coating solution proceeded over night at room temperature. Remaining uncoated spots on the samples were blocked using the synthetic surfactant Pluronic F108. Biotinylated jacalin was added to each vial and the samples were incubated at room temperature. This was followed by addition of streptavidin-peroxidase conjugate. Relative amount of bound mucin was quantified by the addition of substrate, followed by measurement of the degradation product at 405 nm.

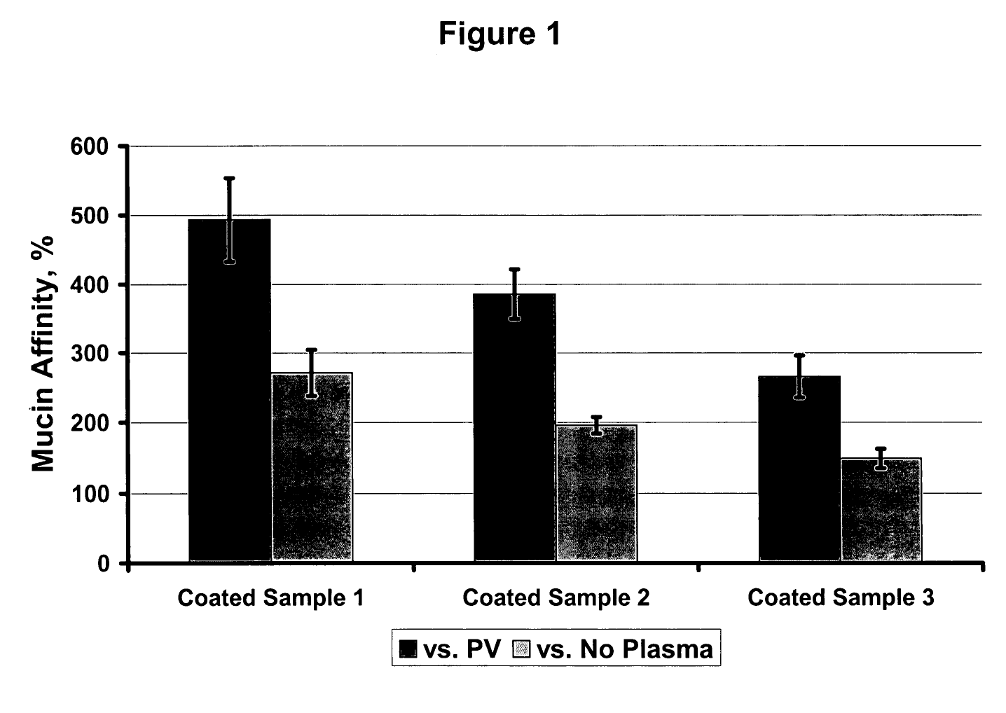

FIG. 1 shows the relative amount of bound mucin compared to both controls for each of the three samples. The absorbance of the enzyme degradation product was measured after a development time of three minutes.

### EXAMPLE 15

### Synthesis of Cationic Polymer

An additional cationic polymer was prepared by the following procedure. To a 1-L 3-neck round bottom flask were added: DMA (39 g, 0.39 moles); DMAPMA-C1 (9 g, 0.029 moles); DMAPMA-C12 (13 g, 0.030 moles); GMA (18 g, 0.13 moles); Vazo 64 AIBN initiator (0.096 g, 0.0006 moles); and tetrahydrofuran (THF, 600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 72 hours. The reaction mixture was then added slowly to 3 L of ethyl ether with good mechanical stirring. The reactive polymer precipitated and gathered on the stir blades as well as in the bottom of the beaker. The stringy solid could not be collected by vacuum filtration. The ether THF mixture was decanted away from the solid and fresh ether was added with stirring. The ether was again removed and polymer was cut away from the stir blades. The beaker containing the polymer mass was then placed in a vacuum oven at 30° C. overnight to remove the ether. The polymer was then broken away from the beaker and ground to a powder, 37.6 g of reactive polymer (47% yield) was isolated. The reactive polymer was placed in a desiccator for storage until use. The high loss of product in this synthesis resulted from an inability to remove the polymer completely from the stirrer and beaker surfaces.

### EXAMPLE 16

### Coating of Contact Lenses with Cationic Polymers

A solution of the subject cationic polymer was prepared by adding the polymer of Example 15 to deionized water at a concentration of 1% (w/v).

Polymacon contact lenses were provided. Polymacon is a hydrogel copolymer composed mainly of 2-hydroxyethyl methacrylate. Lenses were placed in 5 ml of the reactive polymer solution and then put through one 30-minute autoclave cycle (121° C.). The treated lenses were then rinsed twice with HPLC grade water and placed in a saline solution. Lenses were sterilized using a second autoclave cycle and submitted for surface analysis.

### EXAMPLE 17

### Mucin Affinity

In order to assess mucin affinity of the treated polymacon contract lenses of Example 16, an aqueous solution of Alizarin Blue dye was prepared. This dye will bind with hydroxyl groups in a polymacon lens material and with hydroxyl groups in mucin. (1) A polymacon lens with no surface treatment and that was not exposed to the dye was used as a first control. (2) A polymacon lens with no surface treatment was exposed to the dye and used as a second control. This lens turns dark blue due to hydroxyl groups of the polymacon copolymer reacting with the dye. (3) A polymacon lens from Example 16 treated with the cationic polymer did not change color, i.e., it resembled control (1). The resulting surface on this lens is cationic and there are no hydroxyl groups on the lens surface available for interaction with the dye. (4) A polymacon lens from Example 16 was soaked for two minutes in a Bovine mucin solution, rinsed twice with purified water and then exposed to the dye. The lens appeared dark blue, indicating reaction with hydroxyl groups of the mucin retained on the coated lens surface. the surface we have created with the treatment has an affinity for mucin. Lenses were also submitted for XPS and SIMS analyses to confirm the mixed cationic surface.

### EXAMPLE 18

### Synthesis of Cationic Polymer; Coating of RGP Lenses

A further cationic polymer was made, with the following monomers: DMA, 67 mole %; DMAPMA-C1, 5 mole %; DMAPMA-C12, 5 mole %; and GMA, 23 mole %. Rigid, gas permeable (RGP) contact lenses made of Boston XOTM material, a fluorosilicon acrylate copolymer available from Bausch & Lomb Incorporated, were provided.

A solution was prepared with the copolymer given below capable of forming a covalent attachment to the Boston XO surface. The solution was prepared by combining 3.75 grams of the reactive copolymer, 1.5 grams of methyldiethanolamine, and 75 ml of purified water.

The RGP lenses were immersed in 5 ml of the solution and left over night. The samples were then rinsed twice with purified water and padded dry. Samples were analyzed by XPS for surface composition. Lenses treated with the polymer showed significantly reduced fluorine and silicone levels, and increase nitrogen level, at the surface of the substrate, suggesting that the coating was successful.

Examples 19-25 illustrate intermediate polymers that may be used to link various cationic polymers of this invention to a contact lens surface. For example, the intermediate polymers in Examples 19-25 include epoxy functionality that will covalently link to hydroxyl, primary amine or carboxylic acid moieties at the lens surface, and will covalently link to hydroxyl, primary amine or carboxylic acid moieties of the cationic copolymer.

### EXAMPLE 19

### Copolymer of DMA/GMA (86/14 mol/mol)

To a 1 L reaction flask were added distilled N,N-dimethylacrylamide (DMA, 48 g, 0.48 moles), distilled glycidyl methacrylate (GMA, 12 g, 0.08 moles) Vazo 64 initiator (AIBN, 0.1 g, 0.0006 moles) and anhydrous tetrahydrofuran (500 ml). The reaction vessel was fitted with a mechanical stirrer, condenser, thermal controller and a nitrogen inlet. Nitrogen was bubbled through the solution for 15 minutes to remove any dissolved oxygen. The reaction flask was then heated to 40° C. under a passive blanket of nitrogen for 168 hours. The reaction mixture was then added slowly to ethyl ether (1.5 L) with good mechanical stirring. The reactive polymer precipitated and organic solvents were decanted off. The solid was collected by filtration and placed in a vacuum oven to remove the ether leaving 58.2 g of reactive polymer (97% yield). The reactive polymer was placed in a desiccator for storage until use.

### EXAMPLE 20

### Copolymer of DMA/GMA (76/24 mol/mol)

To a 1 L reaction flask were added distilled N,N-dimethylacrylamide (DMA, 42 g, 0.42 moles), distilled glycidyl methacrylate (GMA, 18 g, 0.13 moles) Vazo 64 initiator (AIBN, 0.096 g, 0.0006 moles) and toluene (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 6 L of ethyl ether with good mechanical stirring. The reactive polymer precipitated and was collected by vacuum filtration. The solid was placed in a vacuum oven at 30° C. overnight to remove the ether leaving 46.7 g of reactive polymer (78% yield). The reactive polymer was placed in a desiccator for storage until use.

### EXAMPLE 21

### Copolymer of DMA/GMA (68/32 mol/mol)

To a 1 L reaction flask were added distilled N,N-dimethylacrylamide (DMA, 36 g, 0.36 moles), distilled glycidyl methacrylate (GMA, 24 g, 0.17 moles) Vazo 64 initiator (AIBN, 0.096 g, 0.0006 moles) and toluene (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 6 L of ethyl ether with good mechanical stirring. The reactive polymer precipitated and was collected by vacuum filtration. The solid was placed in a vacuum oven at 30° C. overnight to remove the ether leaving 49.8 g of reactive polymer (83% yield). The reactive polymer was placed in a desiccator for storage until use.

### EXAMPLE 22

### Copolymer of DMA/OFPMA/GMA (84/1.5/14.5 mol/mol/mol)

To a 3000 ml reaction flask were added distilled N,N-dimethylacrylamide (DMA, 128 g, 1.28 moles), 1H,1H,5H-octafluoropentylmethacrylate (OFPMA, 8 g, 0.024 moles), distilled glycidyl methacrylate (GMA, 32 g, 0.224 moles) Vazo-64 initiator (AIBN, 0.24 g, 0.00144 moles) and tetrahydrofuran (2000 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 12 L of ethyl ether with good mechanical stirring. The reactive polymer precipitated and was collected by vacuum filtration. The solid was placed in a vacuum oven at 30° C. overnight to remove the ether leaving 134.36 g of reactive polymer (80% yield). The reactive polymer was placed in a desiccator for storage until use.

### EXAMPLE 23

### Copolymer of DMA/OFPMA/GMA (85/0.18/14.82 mol/mol/mol)

To a 500 ml reaction flask were added distilled N,N-dimethylacrylamide (DMA, 16 g, 0.16 moles), 1H,1H,5H-octafluoropentylmethacrylate (OFPMA, 0.1 g, 0.0003 moles, used as received), distilled glycidyl methacrylate (GMA, 4 g, 0.028 moles) Vazo-64 initiator (AIBN, 0.063 g, 0.00036 moles) and tetrahydrofuran (300 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 3 L of ethyl ether with good mechanical stirring. The reactive polymer precipitated and was collected by vacuum filtration. The solid was placed in a vacuum oven at 30° C. overnight to remove the ether leaving 14.5 g of reactive polymer (69 yield). The reactive polymer was placed in a desiccator for storage until use.

### EXAMPLE 24

### Copolymer of DMA/LMA/GMA (84/1.5/14.5 mol/mol/mol)

To a 1000 ml reaction flask were added distilled N,N-dimethylacrylamide (DMA, 32 g, 0.32 moles), laurylmethacrylate (LMA, 1.5 g, 0.006 moles, used as received), distilled glycidyl methacrylate (GMA, 8 g, 0.056 moles) Vazo-64 initiator (AIBN, 0.06 g, 0.00036 moles) and tetrahydrofuran (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 3 L of ethyl ether with good mechanical stirring. The reactive polymer precipitated and was collected by vacuum filtration. The solid was placed in a vacuum oven at 30° C. overnight to remove the ether leaving 29.2 g of reactive polymer (70% yield). The reactive polymer was placed in a desiccator for storage until use.

### EXAMPLE 25

### Copolymer of NVP/VCHE (85/15 mol/mol)

To a 1 L reaction flask were added distilled N-vinyl-2-pyrrolidinone (NVP, 53.79 g, 0.48 moles), 4-vinylcyclohexyl-1,2-epoxide (VCHE, 10.43 g, 0.084 moles), Vazo 64 (AIBN, 0.05 g, 0.0003 moles) and THF (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 6 L of ethyl ether with good mechanical stirring. The copolymer precipitated and was collected by vacuum filtration. The solid was placed in a vacuum oven at 30° C. overnight to remove the ether leaving 21 g of reactive polymer (a 32% yield). The reactive polymer was placed in a desiccator for storage until use.

Having thus described various preferred embodiment of the invention, those skilled in the art will appreciate that various modifications, additions, and changes may be made thereto without departing from the spirit and scope of the invention, as set forth in the following claims.

We claim:

1. a biomedical device comprising a surface covalently linked to a cationic copolymer.

2. The biomedical device of claim 1, wherein the copolymer comprises cationic monomeric units and hydrophobic monomeric units.

3. The biomedical device of claim 1, which is an ophthalmic device.

4. The biomedical device of claim 3, which is a contact lens.

5. The contact lens of claim 4, wherein the copolymer comprises monomeric units derived from an ethylenically unsaturated monomer containing a quaternary ammonium moiety.

6. The contact lens of claim 5, wherein the copolymer comprises monomeric units derived from at least one of formula (X) or formula (XI):
—(R¹¹)—N⁺(R¹²)(R¹³)(R¹⁴) (X)
wherein: R¹¹ is C(1-3) alkylene; and each of R¹², R¹³ and R¹⁴ is independently methyl or ethyl;
—(R²¹)—N⁺(R²²)(R²³)(R²⁴) (XI)
wherein:
R²¹ is C(1-10) alkylene or fluoroalkylene;
each of R²² and R²³ is independently methyl or ethyl; and
R²⁴ is C(4-22) alkyl or fluoroalkyl.

7. The contact lens of claim 6, wherein the copolymer comprises monomeric units derived from both formula (X) or formula (XI).

8. The contact lens of claim 6, wherein the copolymer comprises monomeric units derived from formula (X) and a non-cationic, ethylenically unsaturated hydrophobic monomer.

9. The contact lens of claim 4, wherein the copolymer further comprises monomeric units derived from an ethylenically unsaturated monomer containing a moiety reactive with complementary reactive functionalities at the lens surface.

10. The contact lens of claim 9, wherein the copolymer is covalently linked to the lens surface with the moiety reactive with the complementary functionality at the lens surface.

11. The contact lens of claim 4, wherein the copolymer comprises: hydrophilic, cationic monomeric units; hydrophobic, cationic monomeric units; and monomeric units derived from an ethylenically unsaturated monomer containing a moiety reactive with complementary reactive functionalities at the lens surface.

12. The contact lens of claim 11, wherein the copolymer further comprises monomeric units derived from a non-cationic, ethylenically unsaturated hydrophilic monomer in an amount sufficient to render the copolymer water soluble.

13. The contact lens of claim 4, wherein the copolymer is covalently bound to the lens surface through primary amine or hydroxyl radicals at the lens surface.

14. The contact lens of claim 4, wherein the copolymer is linked to the lens surface with an intermediate polymer reactive with both the lens surface and the copolymer.

15. The contact lens of claim 14, wherein the intermediate polymer is covalently linked to the lens surface and to the cationic copolymer.

16. The contact lens of claim 15, wherein the copolymer comprises: cationic monomeric units; and monomeric units derived from an ethylenically unsaturated monomer containing a moiety reactive with complementary reactive functionalities of the intermediate polymer.

17. The contact lens of claim 16, wherein the copolymer further comprises monomeric units derived from a non-cationic, ethylenically unsaturated hydrophilic monomer in an amount sufficient to render the copolymer water soluble.

18. The biomedical device of claim 1, wherein the cationic copolymer complexes with mucin.

19. The contact lens of claim 4, wherein the cationic copolymer complexes with epithelial mucin.

20. The contact lens of claim 4, wherein the cationic copolymer is complexed with epithelial mucin.

21. A method comprising covalently linking to a biomedical device surface a cationic copolymer.

22. The method of claim 21, wherein the biomedical device is an ophthalmic device.

23. The method of claim 22, which is a contact lens.

24. The method of claim 23, wherein the copolymer comprises reactive moieties that are covalently linked to complementary reactive functionalities on the lens surface.

25. The method of claim 24, further comprising subjecting the surface of the lens to a plasma treatment that provides the complementary reactive functionalities on the lens surface.

26. The method of claim 24, wherein an intermediate polymer is linked to the lens surface, and the cationic copolymer is linked to the intermediate polymer.

27. The method of claim 26, wherein the intermediate polymer is reactive with reactive functionality on the lens surface and reactive functionalities on the cationic copolymer.

28. The method of claim 27, further comprising subjecting the surface of the lens to a plasma treatment that provides complementary reactive functionalities on the lens surface for the intermediate polymer.

29. The method of claim 23, further comprising placing the contact lens in the eye, whereby mucin complexes with the cationic copolymer.

30. A biomedical device comprising a cationic copolymer covalently linked to a surface of the device, said copolymer complexing with epithelial mucin.

31. The biomedical device of claim 30, which is a contact lens, said copolymer complexing with epithelial mucin.

32. A copolymer comprising: monomeric units derived from an ethylenically unsaturated monomer containing a quaternary ammonium moiety; monomeric units derived from an ethylenically unsaturated hydrophobic monomer; and monomeric units derived from an ethylenically unsaturated monomer containing a moiety reactive with complementary reactive functionalities at a biomedical device surface.
